(19) United States
(12) Patent Application Publication
(10) Pub. No.: US 2004/0157799 A1
(43) Pub. Date: Aug. 12, 2004

(54) Pharmaceutical uses of bisphosphonates

(76) Inventor: John J Seaman, New Hope, PA (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/476,365

(22) PCT Filed: Apr. 30, 2002
(86) PCT No.: PCT/EP02/04771

(51) Int. Cl. A61K 31/675; A61K 31/66

(52) U.S. Cl. 514/89; 514/102

(57) ABSTRACT

A method for the treatment of prostate cancers and other cancers having associated osteoblastic (osteosclerotic) metastases in a patient in need of such treatment comprising administering an effective amount of an N-bisphosphonate, especially zoledronic acid or a salt or any hydrate thereof, to the patient.

This invention relates to bisphosphonates, in particular to new pharmaceuticals uses of bisphosphonates.

Bisphosphonates are widely used to inhibit osteoclast activity in a variety of both benign and malignant diseases which involve excessive or inappropriate bone resorption. These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of osteoporosis, osteopenia, Paget's disease of bone, tumour-induced hypercalcemia (TIH) and, more recently, bone metastases (BM) and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit the formation, recruitment, activation and the activity of osteoclasts.

MM is a plasma-cell malignancy characterized by the proliferation and the accumulation of malignant plasma cells within the bone marrow. The main clinical consequences are lytic bone lesions associated with pathologic fractures and bone pain. These lesions result from an excessive bone resorption, frequently leading to hypercalcemia. Bisphosphonates have been introduced for the long-term treatment of MM in combination with conventional chemotherapy. It has been shown recently that bisphosphonates such as clodronate and pamidronate can reduce the occurrence of skeletal related events such as lytic bone lesions and pathologic fractures and can relieve associated bone pain and improve the quality of life of patients (Laktinen et al. Lancet 1992, 340, 1049-1052; McCloskey et al. B. J. Haematol., 1998, 100, 317-325; and Berenson et al. N. Eng. J. Med. 1996, Vol. 334, No. 8, 488-493). Similar effects have been reported in breast cancer patients treated with bisphosphonates (Hortobagyi G. N. et al. Efficacy of pamidronate in reducing skeletal complications in patients with breast cancer and lytic bone metasases, Protocol 19 Aredia Breast Cancer Study Group, New England Journal of Medicine, 1996; 335:1785-91; Kanis J. A. et al. Clodronate decreases the frequency of skeletal metastases in women with breast cancer, Bone, 1996; 19: 663-7).

Thus bisphosphonates are effective inhibitors of osteoclastic bone resorption and have demonstrated therapeutic efficacy in the treatments of hypercalcemia of malignancy, lytic bone disease associated with multiple myeloma, and mixed lytic and blastic bone metastases associated with breast cancer. However, other cancers, such as prostate cancer have associated skeletal metasases which are predominately osteoblastic (osteosclerotic) in nature and it is not clear whether metastases of these latter cancers will respond similarly to bisphophonate treatment.

Recently it has been reported that bisphosphonate (clodronate, etidronate, alendronate and pamidronate) administration has a favourable effect on bone pain in patients with metastatic prostate cancer carcinoma (Silvio Adami, Cancer 1997; 80: 1674-79). Recently also it has been reported that bisphosphonates inhibit breast and prostate carcinoma cell adhesion to bone in vitro (Boissier et al., Cancer Res; 57: 3890-3894, 1997) and further that pre-treatment of breast and carcinoma cells with bisphosphonates inhibited tumour cell invasion via a direct action on the tumour cells. Yet more recently, it has been reported that in vitro treatment of prostate cancer cell lines with zoledronic acid significantly reduced the growth of the cell lines (Brown et al. Effects of Zoledronate on Prostate Cancer Cells, ASBMR 2000; Lee et al., Bisphosphonate Treatment Inhibits the Growth of Prostate Cancer Cells, Cancer Research, 2000/2001); whereas no significant decrease in tumour volume was detected for subcutaneous prostate cancer cell line tumours treated with zoledronic acid (Corey et al., Effects of Zoledronic Acid on Prostate Cancer in Vitro and in Vivo, Amer. Assoc. Cancer Res. Submitted October 2000).

Hitherto, however, there have been no reports of any bisphophonate having shown clinical efficacy in the treatment of prostate cancer or the bone metastases associated with prostate cancer. We have now shown in a double blind, placebo-controlled clinical study that zoledronic acid (ZOMETA®, Novartis Pharma) demonstrates a statistically significant efficacy benefit over placebo in the treatment of bone metastases in prostate cancer patients.

Accordingly the present invention provides a method for the treatment of prostate cancer in a patient in need of such treatment which comprises administering an effective amount of an N-bisphosphonate to the patient.

The invention further provides use of an N-bisphosphonate in the preparation of a medicament for the treatment of prostate cancer.

The method and use of the invention may be employed for the direct treatment of prostate cancer itself. Thus it is believed that the N-bisphophonates used in the present invention may have a direct effect in vivo on the growth, proliferation or viability of prostate cancer cells, e.g. as inhibitors of prostate cell growth or division or as promoters of prostate cell death (e.g. as apoptosis promoting agents). Conveniently also the invention may be employed for treatment of secondary effects of prostate cancer, including metastases, both soft tissue metastases and bone metastases.

As such It is further believed that the invention may be employed more generally for the treatment of osteoblastic (osteosclerotic) metastases, in particular osteoblastic bone metastases, such as the osteoblastic metastases associated with prostate cancer and similar malignant diseases.

Thus the invention yet further provides use of an N-bisphosphonate to treat osteoblastic metastases associated with malignant diseases or conditions in mammals.

In preferred embodiments the invention provides:

(i) use of an N-bisphosphonate to treat metastases associated with prostate cancer;

(ii) a method for the treatment of metastases associated with prostate cancer in a patient in need of such treatment which comprises administering an effective amount of an N-bisphosphonate to the patient, and

(iii) use of an N-bisphosphonate in the preparation of a medicament for the treatment of metastases associated with prostate cancer.

Efficacy of N-bisphophonate treatment of osteoblastic metastases or prostate cancer metastases according to the invention may be demonstrated by monitoring the occurrence of skeletal related events (SREs) for patients receiving N-bisphosphonate treatment and comparing the results obtained with those obtained for a placebo group; for instance as herein after described in the Clinical Trial Description.

Skeletal related events (SREs) are hereinafter defined in the Clinical Trial Description.

In a particularly preferred embodiment the invention provides:

(i) use of an N-bisphosphonate to reduce SREs associated with metastatic prostate cancer;

(ii) a method for the reduction of SREs associated with metastatic prostate cancer in a prostate cancer patient which comprises administering an effective amount of an N-bisphosphonate to the patient, and

(iii) use of an N-bisphosphonate in the preparation of a medicament for the reduction of SREs associated with metastatic prostate cancer.

In the present description the terms “teatment” or “treat” refer to both prophylactic or preventative treatment as well as curative or disease modifying treatment, including treatment of patients at risk of developing metastases or SREs or suspected to have contracted the disease, e.g. prostate cancer, as well as patients who are ill or have been diagnosed as suffering from a disease or medical condition, e,g, prostate cancer.

For the purposes of the present description an N-bisphosphonate is a compound which in addition to the characteristic geminal bisphosphate moiety comprises a nitrogen containing side chain, e.g. a compound of formula I

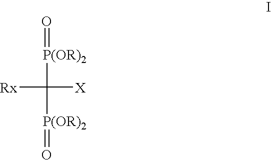

wherein

X is hydrogen, hydroxyl, amino, alkanoyl, or an amino group substituted by C₁-C₄ alkyl, or alkanoyl;

R is hydrogen or C₁-C₄ alkyl and

Rx is a side chain which contains an optionally substituted amino group, or a nitrogen containing heterocycle (including aromatic nitrogen-containing heterocycles), and pharmaceutically acceptable salts thereof or any hydrate thereof.

Thus, for example, suitable N-bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methyl pyridinium salts thereof, for example N-methyl pyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment a particularly preferred N-bisphosphonate for use in the invention comprises a compound of Formula II

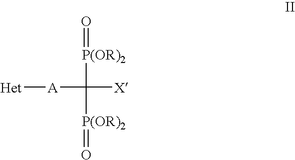

wherein

Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;

A is a straight-chained or branched, saturated or unsaturated hydrocarbon moiety containing from 1 to 8 carbon atoms;

X′ is a hydrogen atom, optionally substituted by alkanoyl, or an amino group optionally substituted by alkyl or alkanoyl radicals, and

R is a hydrogen atom or an alkyl radical,

and the pharmacologically acceptable salts thereof.

In a further embodiment a particularly preferred bisphosphonate for use in the invention comprises a compound of Formula III

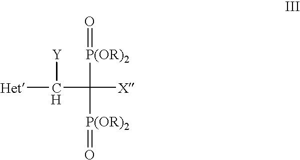

wherein

Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ allyl, C₁-C₄ alkoxy, phenyl, cyclohexyl, cyclohexylmethyl, halogen and amino and wherein two adjacent alkyl substituents of Het can together form a second ring;

Y is hydrogen or C₁-C₄ alkyl;

X″ is hydrogen, hydroxyl, amino, or an amino group substituted by C₁-C₄ alkyl, and

R is hydrogen or C₁-C₄ alkyl;

as well as the pharmacologically acceptable salts and isomers thereof.

In a yet further embodiment a particularly preferred bisphosphonate for use in the invention comprises a compound of Formula IV

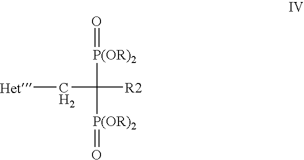

wherein

Het″′ is an imidazolyl, 2H-1,2,3-, 1H-1,2,4 or 4H-1,2,4-triazolyl, tetrazolyl, oxazolyl, isoxazolyl, oxadiazolyl, thiazolyl or thiadiazolyl radical which is unsubstituted or C-mono-or di-substituted by lower alkyl, by lower alkoxy, bx phenyl which may in turn be mnon- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and

R2 is hydrogen, hydroxy, amino, lower alkylthio or halogen,

lower radicals having up to and including 7 C-atoms,

or a pharmacologically acceptable salt thereof.

Examples of particularly preferred N-bisphophonates for use in the invention are:

2-(1-Methylimidazol-2-yl)-1-hydroxyethane-1,1-diphosphonic acid;

2-(1-Benzylimidazol-2-yl)-1-hydroxyethane-1,1-diphosphonic acid;

2-(1-Methylimidazol-4-yl)-1-hydroxyethane-1,1-diphosphonic acid;

1-Amino-2-(1-methylimidazol-4-yl)ethane-1,1-diphosphonic acid;

1-Amino-2-(1-benzylimidazol-4-yl)ethane-1,1-diphosphonic acid;

2-(1-Methylimidazol-2-yl)ethane-1,1-diphosphonic acid;

2-(1-Benzylimidazol-2-yl)ethane-1,1-diphosphonic acid;

2-(Imidazol-1-yl)-1-hydroxyethane-1,1-diphosphonic acid;

2-(Imidazol-1-yl)ethane-1,1-diphosphonic acid;

2-(4H-1,2,4-triazol-4-yl)-1-hydroxyethane-1,1-diphosphonic acid;

2-(Thiazol-2-yl)ethane-1,1-diphosphonic acid;

2-(Imidazol-2-yl)ethane-1,1-diphosphonic acid;

2-(2-Methylimidazol-4(5)-yl)ethane-1,1-diphosphonic acid;

2-(2-Phenylimidazol-4(5)-yl)ethane-1,1-diphosphonic acid;

2-(4,5-Dimethylimidazol-1-yl)-1-hydroxyethane-1,1-diphosphonic acid, and

2-(2-Methylimidazol-4(5)-yl)-1-hydroxyethane-1,1-diphosphonic acid, and pharmacologically acceptable salts thereof.

The most preferred N-bisphosphonate for use in the invention is 2-(imidazol-1yl)-1-hydroxyethane-1,1-diphosphonic acid (zoledronic acid) or a pharmacologically acceptable salt thereof.

Pharmacologically acceptable salts are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts is characterized by having one acidic hydrogen and one pharmaceutically acceptable cation, especially sodium, in each of the phosphonic acid groups.

All the N-bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The N-bisphosphonates may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The N-bisphosphonates can also be used in the form of their hydrates or include other solvents used for their crystallisation.

The N-bisphosphonates are preferably used in the form of pharmaceutical compositions that contain a therapeutically effective amount of active ingredient optionally together with or in admixture with inorganic or organic, solid or liquid, pharmaceutically acceptable carriers which are suitable for administration.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the pharmaceutical compositions are adapted to oral or parenteral (especially intravenous, intra-arterial or transdermal) administration. Intravenous and oral, first and foremost intravenous, administration is considered to be of particular importance. Preferably the N-bisphosphonate active ingredient is in a parenteral form, most preferably an intravenous form.

The particular mode of administration and the dosage may be selected by the attending physician taking into account the particulars of the patient, especially age, weight, life style, activity level, and disease state as appropriate. Most preferably, however, the N-bisphosphonate is administered intravenously.

The dosage of the N-bisphosphonate for use in the invention may depend on various factors, such as effectiveness and duration of action of the active ingredient, mode of administration, warm-blooded species, and/or sex, age, weight and individual condition of the warm-blooded animal.

Normally the dosage is such that a single dose of the bisphosphonate active ingredient from 0.002-20.0 mg/kg, especially 0.01-10.0 mg/kg, is administered to a warm-blooded animal weighing approximately 75 kg. If desired, this dose may also be taken in several, optionally equal, partial doses.

“mg/kg” means mg drug per kg body weight of the mammal—including man—to be treated.

The dose mentioned above—either administered as a single dose (which is preferred) or in several partial doses—may be repeated, for example once daily, once weekly, once every month, once every three months, or less frequently. In other words, the pharmaceutical compositions may be administered in regimens ranging from continuous daily therapy to intermittent cyclical therapy.

Preferably, the N-bisphosphonates are administered in doses which are in the same order of magnitude as those used in the treatment of the malignant diseases classically treated with bisphosphonic acid derivatives, such as tumour-induced hypercalcemia or bone metastases of MM or breast cancer. In other words, preferably the N-bisphosphonic acid derivatives are administered in doses which would likewise be therapeutically effective in the treatment of tumour-induced hypercalcaemia or bone metastases of MM or breast cancer, i.e. preferably they are administered in doses which would likewise effectively inhibit bone resorption and metastases invasion and growth.

Formulations in single dose unit form contain preferably from about 1% to about 90%, and formulations not in single dose unit form contain preferably from about 0.1% to about 20%, of the active ingredient. Single dose unit forms for oral administration such as capsules, tablets or dragées contain e.g. from about 1 mg to about 500 mg of the active ingredient.

Pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Suitable carriers are especially fillers, such as sugars, for example lactose, saccharose, mannitol or sorbitol, cellulose preparations and/or calcium phosphates, for example tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, crosslinked polyvinylpyrrolidone, agar or alginic acid or a salt thereof, such as sodium alginate. Adjuncts are especially flow-regulating agents and lubricants, for example silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethylcellulose phthalate. Colouring substances or pigments may be added to the tablets or dragee coatings, for example for the purpose of identification or to indicate different doses of active ingredient.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intra-arterially, intramuscularly, intraperitoneally, intranasally, intradermally, subcutaneously, preferably intravenously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers. Preferred parenteral forms are intravenous infusion solutions, preferably containg from about 1 mg up to about 20 mg of active substance per unit dose; for instance in an infusion solution volume of from about 5 up to about 200 ml, e.g. for infusion over a period of from about 1 minute up to about 1 hour or more. Such preferred parenteral forms are typically administered at intervals of from about once per week up to about once per 3 months.

The N-bisphosphonates for use in the invention may be administered in combination with other active substances or treatments used for the treatment of prostate cancer and associated metastases.

Thus the invention includes methods for the treatment of prostate cancer patients comprising combined treatment with an N-bisphosphonate and a different anti-prostate cancer agent or an anti-prostate cancer therapy.

The invention further includes a combined composition for the simultaneous, separate or combined treatment of prostate cancer comprising an effective amount of an N-bisphosphonate and an effective amount of a different anti-prostate cancer agent.

Suitable anti-prostate cancer agents include cytotoxic chemotherapeutic agents, e.g. doxorubicin, danorubicin etc., Cisplatin etc., Taxol, hormonal agent, e.g. LHRH and analogues thereof, steroids and biological response modifier agents.

Suitable anti-prostate cancer therapy includes radiation therapy to treat extra-skeletal and/or skeletal tumor sites.

Other therapeutic agents which may be used in combination with N-bisphosphonates in prostate cancer therapy are hereinafter described in the Clinical Trial Description.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following Examples illustrate the invention described hereinbefore.

In the following Examples the term “active ingredient” is to be understood as being any one of the N-bisphosphonic acid derivatives mentioned above as being useful according to the present invention.

### EXAMPLES

### Example 1

Capsules containing coated pellets of active ingredient, for example, disodium pamidronate pentahydrate, as active ingredient:

Core pellet:

| | |
|---|---|
| active ingredient (ground) | 197.3 mg |
| Microcrystalline cellulose | 52.7 mg |
| (Avicel ® PH 105) | |
| | 250.0 mg |

+Inner coating:

| | |
|---|---|
| Cellulose HP-M 603 | 10.0 mg |
| Polyethylene glycol | 2.0 mg |
| Talc | 8.0 mg |
| | 270.0 mg |

+Gastric juice-resistant outer coating:

| | |
|---|---|
| Eudragit ® L 30 D (solid) | 90.0 mg |
| Triethyl citrate | 21.0 mg |
| Antifoam ® AF | 2.0 mg |
| Water | |
| Talc | 7.0 mg |
| | 390.0 mg |

A mixture of active ingredient, e.g. disodium pamidronate, with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit® L 30 D, triethyl citrate and Antifoam® AF. The coated pellets are powdered with talc and filled into capsules (capsule size 0) by means of a commercial capsule filling machine, for example Höfliger and Karg.

### Example 2

Monolith adhesive transdermal system, containing as active ingredient, for example, 1-hydroxy-2-(imidazol-1-yl)-ethane-1,1-diphosphonic acid:

Composition:

| | |
|---|---|
| polyisobutylene (PIB) 300 | 5.0 g |
| (Oppanol B1, BASF) | |
| PIB 35000 | 3.0 g |
| (Oppanol B10, BASF) | |
| PIB 1200000 | 9.0 g |
| (Oppanol B100, BASF) | |
| hydrogenated hydrocarbon resin | 43.0 g |
| (Escorez 5320, Exxon) | |
| 1-dodecylazacycloheptan-2-one | 20.0 g |
| (Azone, Nelson Res., Irvine/CA) | |
| active ingredient | 20.0 g |
| Total | 100.0 g |

Preparation:

The above components are together dissolved in 150 g of special boiling point petroleum fraction 100-125 by rolling on a roller gear bed. The solution is applied to a polyester film (Hostaphan, Kalle) by means of a spreading device using a 300 mm doctor blade, giving a coating of about 75 g/m². After drying (15 minutes at 60° C.), a silicone-treated polyester film thickness 75 mm, Laufenberg) is applied as the peel-off film. The finished systems are punched out in sizes in the wanted form of from 5 to 30 cm² using a punching tool. The complete systems are sealed individually in sachets of aluminised paper.

### Example 3

Vial containing 1.0 mg dry, lyophilized 1-hydroxy-2-(imidazol-1-yl)ethane-1,1-diphosphonic acid (mixed sodium salts thereof). After dilution with 1 ml of water, a solution (concentration 1 mg/ml) for i.v. infusion is obtained.

Composition:

| | | |
|---|---|---|
| active ingredient (free diphosphonic acid) | 1.0 | mg |
| mannitol | 46.0 | mg |
| Trisodium citrate × 2 H₂O ca. | 3.0 | mg |
| water | 1 | ml |
| water for injection | 1 | ml. |

In 1 ml of water, the active ingredient is titrated with trisodium citrate x 2H₂O to pH 6.0. Then, the mannitol is added and the solution is lyophilized and the lyophilisate filled into a vial.

### Example 4

Ampoule containing active ingredient, for instance disodium pamidronate pentahydrate dissolved in water. The solution (concentration 3 mg/ml) is for i.v. infusion after dilution.

Composition:

| | | |
|---|---|---|
| active ingredient | 19.73 | mg |
| ( [symbol] 5.0 mg of anhydrous active ingredient) | | |
| mannitol | 250 | mg |
| water for injection | 5 | ml. |

### Clinical Trial Description

1. Study Objectives

The primary objective of this study is to assess the efficacy of zoledronic acid treatments (4 or 8 mg) in addition to antineoplastic therapy, compared to antineoplastic therapy alone to prevent skeletal-related events in prostate cancer patients with a history of metastatic bone disease who have developed biochemical progression of disease [i.e., an increasing serum PSA level] while on first-line hormonal therapy for metastatic disease. Skeletal-related events (SREs) are defined as pathologic bone fracture events, spinal cord compression events, surgery to bone, radiation therapy to bone (including the use of radioisotopes) and a change of antineoplastic therapy to treat bone pain. Thus, SREs are the primary end-points in this study. The key efficacy endpoints are the proportion of patients having at least one SRE and the time to first SRE.

Secondary objectives are to assess the effect of zoledronic acid therapy on pain scores, analgesic use, performance status, Quality of Life scores, time to termination of active study participation, and to assess the safety and tolerability of zoledronate. Changes in bone resorption and formation markers are also evaluated. Bone mineral density measurements are evaluated in patients at selected clinical centers. In addition, time to progression of disease in bone and time to overall progression of disease are evaluated.

Tertiary objectives are to measure health care utilization and productivity loss data.

2. Investigational Plan

2.1. Overall Study Design

This study is an international, multicenter, randomized, double-blind, placebo-controlled, parallel study. The patient population of this study consists of prostate cancer patients with a history of metastatic bone disease who have a rising serum PSA concentration (see Section 2.3.2.) despite treatment with first-line hormonal therapy for metastatic disease. The rising serum PSA level is documented by three consecutively rising serum PSA measurements (i.e., the third PSA level is to be>the second PSA level which is >the first PSA level which is >the nadir serum PSA concentration achieved during first-line hormonal therapy for metastatic prostate cancer), each separated from the other by at least two weeks. The rising serum PSA level represents an “early” event in the development of progressive metastatic disease. Although progressive lesions may have appeared on bone radiographic studies (bone scan and/or bone survey) without affecting a patient's study eligibility, patients who have developed bone pain since their best response to first-line hormonal therapy for metastatic disease prior to study entry (visit 2—date of randomization and initial study drug treatment) are excluded from the study.

In addition, an alteration of the first-line hormonal regimen prior to Visit 1 (the screening visit) is also an exclusion criterion. Otherwise, a patient's antineoplastic therapy regimen may be changed during the study (including Visit 1 through Visit 34) at the discretion of the treating physician except for the administration of cytotoxic chemotherapy prior to and including Visit 2 (subsequent use of cytotoxic chemotherapy during the study is permitted). Other exclusion criteria include (see section 2.3.2.) a serum testosterone level at Visit 1 above the castrate range (≧50 ng/ml), the use of radiation therapy to bone within three months of Visit 2 (includes the use of radioisotopes), and prior or current (up to and including Visit 2) use of cytotoxic chemotherapy (the use of cytotoxic chemotherapy is permitted during the study following Visit 2 at the discretion of the treating physician).

Patients are randomized in a double-blind fashion to receive either zoledronate 4 mg intravenously, or zoledronate 8 mg intravenously, or a placebo intravenous infusion every three weeks in addition to their antineoplastic therapy. The randomized treatment assignment ratio is to be 1:1:1. In addition, all patients are to receive 500 mg of calcium orally and a multivitamin tablet (containing 400-500 I.U. of vitamin D) daily throughout the study.

The occurrence of skeletal-related events (SRE's) is collected throughout the trial for each patient in order to determine the proportion of patients experiencing at least one SRE, the time to the first SRE, and the skeletal morbidity rate (see section 4). Time to progression of disease in bone is assessed centrally (central radiologist) by review of serial bone radiographic studies. Time to overall progression of disease is determined by the patient's treating physician: by evaluating the central assessments (central radiologist) of serial bone radiographic studies; by evaluating the central assessments (central radiologist) of appropriate serial radiographic studies of non-skeletal tumor sites, if present; by evaluating serial serum PSA levels; and by evaluating serial patient weight measurements. Quality of Life, performance status, health care utilization and productivity loss data, and pain, and analgesic use scores are determined serially throughout the study. This information is also collected for those patients who discontinue active therapy within 24 months from the date of randomization into the study. Information about adverse events which occur during the patient's clinical course is collected throughout the study.

Study procedures begin with a pre-randomization screening visit (Visit 1) which may last for up to 14 days in order to perform baseline evaluations. Randomization occurs at Visit 2 prior to study drug administration after the eligibility criteria have been met and all screening evaluations have been completed. The study is made up of two phases, phase 1, the Efficacy and Safety Phase, and phase 2, the Extension Phase. The primary efficacy analysis is performed at the completion of phase 1, the Efficacy and Safety Phase, which consists of 60 weeks (20 cycles) of study treatment. Phase 2 consists of an additional 36 weeks (12 cycles) of study therapy. The primary purpose of phase 2 is to obtain long-term zoledronate treatment safety and survival data, however efficacy data will continue to be collected. At least five hundred and fifty patients are enrolled in order to obtain 519 patients (173 patients per treatment arm) who meet the protocol entry criteria. No interim analyses are planned.

Patients are not discontinued from the study solely because of the occurrence of a skeletal related event or progression of disease during the study because the study is designed to evaluate the total number of skeletal events that occur over the entire duration of the study (24 months). In addition, the antineoplastic therapy may be changed without causing the patients to be terminated from the study. Patients who discontinue therapy remain on study for collection of skeletal-related events, disease progression, antineoplastic therapy, healthcare utilization and productivity losses, Quality of Life, pain, and analgesic score data.

Survival data is collected for each patient randomized into the study and collected at 6 month intervals after the patient is discontinued from the study until 24 months after last patient first visit for the study.

The following tables summarize the study design:

Schematic Design Diagram* - Phase 1 (Safety and Efficacy)

| | | | Final Evaluation Phase |
|---|---|---|---|
| | | | 1 and First |
| | | Randomized Treatment | Randomized |
| Period | Screening | and Evaluation | Treatment Phase 2 |
| Visit | 1 | 2 3 4 5 6 7 8 9 10 11 12 | 22 |
| | | 13 14 15 16 17 | |
| | | 18 19 20 21 | |
| Week | −2 weeks | 0 3 6 9 12 15 18 21 24 | 60 |
| | | 27 30 33 36 39 42 45 | |
| | | 48 51 54 57 | |
| Treatment | none | Zoledronate 4 mg q | Zoledronate 4 mg q 3 |
| | | 3 weeks | weeks |
| | | Zoledronate 8 mg q | Zoledronate 8 mg q 3 |
| | | 3 weeks | weeks |
| | | Placebo q 3 weeks | Placebo q 3 weeks |

Schematic Design Diagram - Phase 2 (Extension)

| Period | Randomized Treatment and Evaluation | Evaluation |
|---|---|---|
| Visit | 23 24 25 26 27 28 29 30 31 32 33 | 34 |
| Week | 63 66 69 72 75 78 81 84 87 90 93 | 96 |
| Treatment | Zoledronate 4 mg q 3 weeks | none |
| | Zoledronate 8 mg q 3 weeks | |
| | Placebo q 3 weeks | |

*Following Visit 2, study visits are to be made on the designated study day with an error of not more than −3 to +7 days.

Duration of Study

Time permitted for patient enrollment: 12 months

Duration of individual patient's participation:

15 months (60 weeks) Phase 1

9 months (36 weeks) Phase 2

Total duration of treatment: 24 months (96 weeks)

Total duration of study: 36 months

2.2. Study Population

2.2.1. Inclusion and Exclusion Criteria

Inclusion Criteria

Signed informed consent.

Patients 18 years of age or greater.

A histologically confirmed diagnosis of carcinoma of the prostate.

Patients must have or have had objective evidence of metastatic disease to bone. Objective evidence of metastatic bone disease is defined as multiple foci (>3) of increased activity on bone scan. If there are ≦3 foci of increased activity on bone scan, additional radiographic or biopsy studies are required to confirm the presence of osteoblastic or osteolytic malignant bone lesions. Patients who have achieved a complete response to first-line hormonal therapy and their current bone scan is normal are still eligible to enroll in this study as long as bone metastases have been documented previously during the patient's clinical course.

Patients must demonstrate biochemical progression of disease despite therapy with first-line hormonal treatments (medical or surgical castration). Biochemical disease progression is defined as follows:

Three consecutively rising serum PSA measurements, each separated from the other by at least two weeks. The third serum PSA measurement must be >0.4 ng/ml.

First-line hormonal therapy for metastatic disease is defined as follows:

The initial hormonal regimen utilized to treat metastatic prostate cancer. Hormonal therapy given in the neoadjuvant or adjuvant setting at a time when there is no clinical evidence of metastatic disease will not be considered as first-line hormonal therapy for metastatic disease for the purposes of this study.

The patient must have an ECOG performance status of 0, 1, or 2.

Exclusion Criteria

Bone pain due to metastatic bone disease that has developed since the best response to first-line hormonal therapy for metastatic disease.

Previous or current (prior to and including Visit 2) treatment with cytotoxic chemotherapy (subsequent use of cytotoxic chemotherapy during the study is permitted).

Alteration of the first-line hormonal therapy to a second-line hormonal regimen prior to Visit 1 (subsequent alteration of the patient's hormonal therapy during Visit 1 or throughout the study is not an exclusion criterion or a protocol violation).

Serum testosterone level (at Visit 1) elevated above the castrate range (≧50 ng/ml).

Radiation therapy to bone (including radioisotopes) within 3 months prior to Visit 2.

Previous treatment with a bisphosphonate agent.

Treatment with calcitonin, mithramycin, or gallium nitrate within 2 weeks prior to the date of randomization (Visit 2).

Use of other investigational drugs (drugs not marketed for any indication) within 30 days prior to the date of randomization (Visit 2).

History of noncompliance to medical regimens and patients who are considered potentially unreliable or incapable of giving informed consent as judged by the investigator.

Serum creatinine concentration>3.0 mg/dL (265 μmol/L).

Corrected (adjusted for serum albumin) serum calcium concentration<8.0 mg/dL (2.00 mmol/L) or ≧11.6 mg/dL (2.90 mmol/L).

History of any other neoplasm within the past five years except for nonmelanomatous skin cancer.

Patients with evidence in the six months prior to randomization of severe cardiovascular disease (defined as uncontrolled congestive heart failure), hypertension refractory to treatment, or symptomatic coronary artery disease.

2.3. Treatments

2.3.1. Investigational Drug and Reference Therapy

Patients receive zoledronate or placebo as a 5-minute intravenous infusion every 3 weeks for 24 months.

Patients will receive 500 mg of calcium by mouth with food daily in the evening during the study. The calcium will be supplied by the investigative site as an open-label drug. Each package will have a label affixed to it with instructions to “take one dose daily in the evening with food”. Patients will also receive one multivitamin tablet by mouth daily in the morning during the study supplied by the investigative site as an open-label drug. Each package will have a label affixed to it with the instructions to “take one dose daily in the morning with food”.

Study drug supplies (zoledronate) will be shipped to the pharmacist at each center. Drug will be packaged in an open-labeled fashion. Medication labels will comply with the legal requirements of each country and be printed in the local language. They will supply no information about the patient. The storage conditions for study drug will be described on the medication label.

The name and dose of drug will be provided on each vial.

Zoledronate will be supplied in 4 mg lyophilized vials (4000 ug).

The study drug will be stored in a locked area at each center until it is returned to Novartis at the end of the study. The pharmacist will be responsible for the preparation of study drug. Documentation of study drug administration and amount received at each visit will be maintained for every patient.

If reconstituted zoledronate solutions cannot be used immediately solutions must be refrigerated at temperatures between 36-46° F. (2-8° C.) and can be used for up to eight hours.

As zoledronate may bind to glass, the study-drug solutions should be prepared in plastic syringes, bags, and tubes. The zoledronate is to be given intravenously to each patient as a 5-minute infusion. Each 4 mg vial of zoledronate is to be reconstituted with 5 ml of sterile water for injection. The appropriate volume of reconstituted zoledronate is to be mixed with an appropriate volume of physiologic (0.9%) normal saline so that the total volume infused is 50 ml. Each patient will receive the same study drug and dose throughout the study (Visits 2-33) depending on the treatment group to which they are assigned.

The treatment groups are:

zoledronate 4 mg in 50 ml normal saline intravenous infusion every 3 weeks plus calcium 500 mg taken by mouth with food (daily) and one multivitamin tablet by mouth daily.

zoledronate 8 mg in 50 ml normal saline intravenous infusion every 3 weeks plus calcium 500 mg taken by mouth with food (daily) and one multivitamin tablet by mouth daily.

placebo 50 ml normal saline intravenous infusion every 3 weeks plus calcium 500 mg taken by mouth with food (daily) and one multivitamin tablet by mouth daily.

3-Compartment, double-blind study

| | | | | Normal | |
|---|---|---|---|---|---|
| | | | | Saline | Total |
| | | | Reconstituted | Volume | Volume |
| Medication | Visit | Total # Vials | Volume | Added | Infused |
| Zoledronate 4 mg | 2-33 | 1 vial of 4.0 mg/vial | 5.0 ml | 45 ml | 50 ml |
| in NS I.V. | | reconstitute | | | |
| every 3 weeks | | with sterile | | | |
| | | water for | | | |
| | | injection 5 ml/vial | | | |
| Zoledronate 8 mg | 2-33 | 2 vials of 4.0 mg/vial | 10 ml | 40 ml | 50 ml |
| in NS I.V. every 3 | | reconstitute | | | |
| weeks | | with sterile | | | |
| | | water for | | | |
| | | injection 5 ml/vial | | | |
| Placebo in NS I.V. | 2-33 | N/A | N/A | 50 ml | 50 ml |
| q 3 wks | | | | | |

Initially a 5-minute 50 ml infusion was used; this was changed to a 15-minute 100 ml infusion to increase renal safety. A further protocol amendment reduced the dose of the zoledronic acid 8 mg group to 4 mg. Patients already receiving 8 mg had their dose reduced at subsequent visits and patients newly randomized to the 8 mg group only received 4 mg.

2.4. Concomitant Therapy

The following treatments are allowed:

Standard antineoplastic therapies including marketed cytotoxic chemotherapy agents, hormonal agents, steroids and biologic response modifier agents.

Standard radiation therapy to treat extra-skeletal and/or skeletal tumor sites.

Standard marketed cytokine/colony stimulating factor agents.

Marketed drugs/therapies except those that would be expected to affect osteoclast activity (e.g. calcitonin, mithramycin, gallium nitrate, any other bisphosphonate). Thus, if a treating physician determines that a study patient's medical condition (e.g., an osteoporosis or tumor-induced hypercalcemia) requires the use of an inhibitor of osteoclastic bone resorption, then the patient is to be discontinued from active study participation and is to be followed for skeletal-related event data (see Section 2.3.3.).

Corticosteroid therapy utilized to prevent/treat chemotherapy-induced nausea/vomiting.

Corticosteroid therapy for spinal cord compression or other recognized indications.

3. RESULTS

At least one skeletal-related event occurred in 33.2% and 38.5% of patients in the zoledronic acid 4 mg (N=214) and 8/4 mg (N=221) groups compared with 44.2% in the placebo group (N=208, p=0.021 and 0.222 vs. placebo). Pathologic fractures occurred in 13.1% and 14.9% of patients in the zoledronic acid 4 mg and 8/4 mg groups and in 22.1% of patients in the placebo group (p=0.015 and p=0.054 vs. placebo). Median time to the first skeletal-related event was not reached in the zoledronic acid 4 mg group, and was 363 and 320 days in the 8/4 mg and placebo groups, respectively (p=0.011 and 0.491 vs. placebo). Zoledronic acid 4 mg given as a 15-minute infusion was well-tolerated.

Zoledronic acid 4 mg 15-minute infusions every 3 weeks significantly reduce skeletal-related events in patients with metastatic prostate cancer refractory to hormonal therapy.

1. A method for the treatment of prostate cancer in a patient in need of such treatment which comprises administering an effective amount of an N-bisphosphonate to the patient.

2. Use of an N-bisphosphonate in the preparation of a medicament for the treatment of prostate cancer.

3. Use of an N-bisphosphonate to treat osteoblastic metastases associated with malignant diseases or conditions in mammals.

4. Use of an N-bisphosphonate in the preparation of a medicament for the treatment of osteoblastic metastases associated with malignant diseases or conditions in mammals.

5. A method for the treatment of metastases associated with prostate cancer in a patient in need of such treatment which comprises administering an effective amount of an N-bisphosphonate to the patient.

6. Use of an N-bisphosphonate in the preparation of a medicament for the treatment of metastases associated with prostate cancer in a patient in need of such treatment.

7. A method for the reduction of SREs associated with metastatic prostate cancer in a prostate cancer patient which comprises administering an effective amount of an N-bisphosphonate to the patient.

8. Use of an N-bisphosphonate in the preparation of a medicament for the reduction of SREs associated with metastatic prostate cancer in a prostate cancer patient.

9. A method according to claim 1 or a use according to claim 2 in which the N-bisphophonate is a compound of formula I 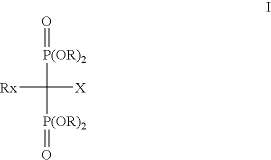
wherein
X is hydrogen, hydroxyl, amino, alkanoyl, or an amino group substituted by C₁-C₄ alkyl, or alkanoyl;
R is hydrogen or C₁-C₄ alkyl and
Rx is a side chain which contains an optionally substituted amino group, or a nitrogen containing heterocycle (including aromatic nitrogen-containing heterocycles),
or pharmaceutically acceptable salts thereof or any hydrate thereof.

10. A method or use according to claim 9 in which the N-bisphohonate is one of the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methyl pyridinium salts thereof, for example N-methyl pyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

11. A method according to claim 1 or use according to claim 2 in which the N-bisphosphonate for use in the invention is 2-(imidazol-1yl)-1-hydroxyethane-1,1-diphosphonic acid (zoledronic acid) or a pharmacologically acceptable salt thereof.

12. A method for the treatment of prostate cancer patients comprising combined treatment with an N-bisphosphonate and a different anti-prostate cancer agent or an anti-prostate cancer therapy.
